(19) United States
(12) Patent Application Publication
(10) Pub. No.: US 2006/0021078 A1
(43) Pub. Date: Jan. 26, 2006

(54) Lettuce variety nasimento

(76) Inventor: Johannes Theodorus van Schijndel, Gravenzande (NL)

Correspondence Address: BAYER CROPSCIENCE LP;Patent Department, 100 BAYER ROAD, PITTSBURGH, PA, 15205-9741, US

(21) Appl. No.: 11/178,913

(22) Filed: Jul. 11, 2005

(51) Int. Cl. A01H 1/02; A01H 5/00; A01H 1/00

(52) U.S. Cl. 800/260; 800/298

(57) ABSTRACT

The lettuce variety Nasimento is disclosed. The invention relates to seeds, plants, plant cells, plant tissue and harvested products as well as to hybrid lettuce plants and seeds obtained by repeatedly crossing plants of Nasimento with other lettuce plants. The invention also relates to plants and varieties produced by the method of essential derivation from plants of Nasimento and to plants of Nasimento reproduced by vegetative reproduction methods including but not limited to tissue culture of regenerable cells or tissue from Nasimento.

### FIELD OF THE INVENTION

The invention is in the field of pant breeding and relates to a variety of lettuce designated as Nasimento and its essentially derived varieties and the hybrid varieties obtained by crossing Nasimento as a parent line with plants of other varieties or parent lines.

### BACKGROUND OF THE INVENTION

The invention is in the field of plant breeding in the vegetable crop lettuce, botanical species Lactuca sativa. Lettuce is an important vegetable crop for salads and food dressing. Its regular sexual reproduction develops via obligatory self pollination and fertilization, which facilitates the preservation of the plant characteristics during breeding and seed production. The agronomic characteristics which are necessary for producing a healthy, good yielding and leafy crop are to be combined with characteristics or traits for a high seed production of an excellent quality. Due to the complexity of the genetic basis—the interactions between genes, dependent on the position of the genes in the genome, and the interaction between the genetic composition of the genotype and the environment,—the expression of said genotype in the phenotype,—the eventual plant variety—, is unpredictable for the person or the ordinary plant breeder, skilled in the art. The breeder can only apply his skills on the phenotype and not, or in a limited way, on the level of the genotype. Due to this phenomenon a particular plant breeder cannot breed the same variety twice using the same breeding parents and the same methodology. Each variety contains a unique combination of—mostly—known phenotypic characteristics. By carefully choosing the breeding parents, the breeding and selection methods, the testing layout and testing locations, the breeder can aim at a particular variety type. Before the invention—the new variety—can be released in practice, it is often tested in special comparative trials with other existing varieties, in order to determine whether the new invention meets the required expectations.

### SUMMARY OF THE INVENTION

The invention relates to seeds, plants, plant cells, parts of plants and harvested products as well as to hybrid lettuce plants and seeds produced by repeatedly crossing plants of Nasimento with other lettuce plants. The invention relates also to plants and varieties produced by the method of essential derivation from plants of Nasimento and to plants of Nasimento reproduced by a method of vegetative reproduction including but not limited to regeneration of embryogenic cells or tissue of Nasimento.

### DESCRIPTION OF THE INVENTION

The invention has been obtained by a general breeding process comprising the following steps (for reference see chapter 11 of Briggs and Knowles 1967, titled “Breeding self-pollinated crops by hybridization and pedigree selection”):

Parent plants, which have been selected for good agronomic traits, including resistance to aphids, in particular the aphid Nasonovia ribisnigri as described in the U.S. Pat. No. 5,977,443 and good seed production traits are manually crossed in different combinations. The resulting F 1 (Filial generation 1) plants are self fertilized and the resulting F2 generation plants, which show a large variability on account of optimal gene recombination and segregation, are planted in a selection field.

These F2 plants are observed during the growing season for plant health (pest resistance, including resistance to Nasonovia ribisnigri and disease resistance), tip burn sensitivity, growth vigor, plant type, leafiness and yield. Seeds of the selected plants are harvested and in the next growing seasons this procedure is repeated, whereby the selection and testing units increase from individual plants in the F2, to multiple plant containing ‘lines’ (descending from one mother plant) in the F5. The number of units decreases from approximately 200 plants in the F2 to approximately 3-5 lines in the F5 by selecting about 20-30% of the units in each selection cycle.

The increased size of the units, whereby more seed per unit is available, allows the selection and testing in replicated trials on more than one location with a different environment and a more extensive and accurate analysis of the results.

The lines become genotypically more homozygous and phenotypically more homogeneous or uniform by selecting similar plant types within a line and by discarding plants with different characteristics, the so called off-types, from the very variable F2 on to the final, very uniform F7 or F8 generation. The seeds produced by the plants of this final generation form eventually the basis for the new variety and for the further generations of seed production.

Depending on the intermediate results the plant breeder may decide to vary on the procedure as described above: e.g. accelerating the process by testing a particular line earlier or by retesting a particular line for another year. He may also select plants for further crossing with existing breeding-parent plants or with other plants resulting from the current selection procedure.

By the method of recurrent backcrossing, well known in the field of the invention (e.g. see Briggs and Knowles 1967 in chapter 13, titled “The backcross method of breeding”), the breeder can introduce a specific trait into an existing valuable line or variety, while preserving the original characteristics of this line or variety. In this crossing method the valuable parent is recurrently used to cross it at least for 2 times with each resulting F1, followed by selection of the recurrent parent plant type, possibly with the aid of molecular markers, until the genotype and the phenotype of the resulting F1 plants are close to that of the recurrent parent. The use of molecular markers in the selection process has significantly reduced the number of backcrosses, necessary to regain the type of the recurrent parent: from 4 or 5 crosses in the past to 2 or 3 crosses nowadays, depending on the crop. In the case of lettuce, 2 backcrosses with subsequent marker aided selection are sufficient to recover 95% of the genotype of the recurrent parent. See for reference P. Stam, 2003.

This method of recurrent backcrossing results eventually in an essentially derived variety, which is predominantly derived from the recurrent parent or initial variety and retains the original phenotypic characteristics of the recurrent or initial variety that are the expression of the genotype of the initial variety, except for the characteristics—e.g. the desired trait or traits from the donor parent—that have been changed due to the method of essential derivation.

Besides the introduction of a desired trait this method can also be used to get as close as possible to the genetic composition of an existing successful variety by changing a trait,—which can be any phenotypic trait—, that renders the essentially derived variety distinct from the recurrent or initial parent—, with the intention to profit from the qualities of that successful initial variety.

The genetic conformity between two varieties can be determined by a set of molecular markers and by using appropriate statistical methods as mentioned by van Eeuwijk and Law 2004 (see list of references). As used herein, if the Jacard coefficient between the putative essentially derived variety and the initial variety is 96% or higher and the initial variety has been predominantly used in the creation of the putative essentially derived variety, then that variety is essentially derived from the initial variety and is included within the scope of this invention.

Except via recurrent backcrossing as described in the preceding paragraphs, such essentially derived variety, may be obtained by other breeding methods as for instance, but not limited to, the doubling of chromosomes by colchicine application.

The product of essential derivation is an essentially derived variety, which variety is—except for the one, or two, or three, or four, or five, phenotypic, distinctive characteristics,—which characteristics are different as the result of the act of derivation—characterized by the same expression of the characteristics in its phenotype as in the phenotype of the initial variety, which same expression results from the genotype of the essential derived variety, which genotype may be identical,—as it can in many cases not be differentiated from the genotype of the initial variety with the current molecular marker methods—, or is almost identical or very similar to the genotype of the initial variety. Plants of the essentially derived variety can be used to repeat the process of essential derivation. The-resulting variety of this repeated process is also a variety essentially derived from the said initial variety as it retains the expression of the phenotypic characteristics of the initial variety, except for the one, or two, or three, or four, or five, phenotypic, distinctive characteristics, which characteristics are different as the result of the act of derivation.

One embodiment of this invention, the lettuce variety “Nasimento” as used herein, refers to plants, seeds, plant parts, cells, or tissue having the characteristics of the lettuce variety of which seeds are deposited under NCIMB accession number 41293.

Another embodiment of this invention relates to seeds, plants, plant parts, cells and tissue of lettuce varieties that are essentially derived from Nasimento—by, but not limited to selection of a natural or induced mutant or of a (somaclonal) variant or by the method of repeated back crossing or by genetic engineering—, being essentially the same as this invention by expressing the unique combination of characteristics of Nasimento, except for one, or two, or three, or four, or five, characteristics being different from the characteristics of Nasimento as a result of the act of derivation.

Another embodiment of this invention is the reproduction of plants of Nasimento by the method of tissue culture, from any regenerable plant tissue obtained from plants of this invention. Plants so reproduced express the specific combination of characteristics of this invention and fall within its scope. During one of the steps of the reproduction process via tissue culture somaclonal variants may occur, which can be selected as being distinct from this invention, but still fall in the scope of this invention as being essentially derived from this invention.

Again another embodiment of this invention is the production of a hybrid variety by the well known method of repeatedly crossing plants of Nasimento with plants of a different variety or varieties or with plants of a non-released line or lines. In practice 3 different types of hybrid varieties may be produced (see for reference chapter 18 of Briggs and Knowles 1967 titled “Hybrid varieties”): The “single cross” hybrid produced by 2 different lines, the “three way hybrid”, produced by 3 different lines such that first the single hybrid is produced by using 2 out of the 3 lines followed by crossing this single hybrid with the third line, and the “four way hybrid” produced by 4 different lines such that first 2 single hybrids are produced using the lines 2 by 2, followed by crossing the 2 single hybrids so produced. Each single, three way or four way hybrid variety so produced and using Nasimento as one of the parent lines contains an essential contribution of Nasimento to the resulting hybrid variety and falls in the scope of this invention.

Lettuce can be grown in different ways. The most common way is as spaced plants in order to obtain full grown plants to use the leaves in salads or for decoration in several dishes. A specific way of growing lettuce in order to produce small lettuce leaflets is the seeding at high density. The resulting plantlets are capable to express all the phenotypic characteristics of Nasimento, resulting from the genotype of Nasimento and hence these plantlets fall within the scope of this invention.

All literature and references cited are incorporated by reference in this description.

### EXAMPLE 1

Seeds were obtained from plants finally selected in the process of breeding the new variety “Nasimento”. A representative sample of at least 2500 seeds of Nasimento has been deposited under the Budapest Treaty on the International Recognition of the Deposit of Micro-organisms for the Purposes of Patent Procedure at the NCIMB Ltd. (Ferguson Building, Craibstone Estate, Bucksbum, Aberdeen AB21 9YA, UK) under NCIMB Accession number 41293, on Jun. 1, 2005.

Applicants hereby waive any restrictions on the public availability of the deposited material from the NCIMB, once a U.S. patent has been granted on this application. However, Applicants have no authority to waive any restrictions imposed by law on the transfer, importation or (commercial) use of biological material. The Applicant does not waive any infringement of its rights granted under a patent on this application, or under the Plant Variety Protection Act (7 USC 2321 et seq.).

Seeds of Nasimento were planted in a field trial, together with seeds of lettuce variety Darkland as a reference variety. The results as shown in Table 1 were obtained from an analysis of the data from this trial.

TABLE 1
Description of Nasimento in comparison to the similar lettuce variety
Darkland, according to the protocol CPVO - TP 13/1 of the Com-
munity Plant Variety Office, Angers, France.

| lDescription according to the CPVO protocol | Nasimento | Darkland |
|---|---|---|
| Seed: colour | 1 | 1 |
| Seedling: anthocyanin coloration | 1 | 1 |
| Seedling: size of cotyledon (fully developed) | 3 | 3 |
| Seedling: shape of cotyledon | 3 | 3 |
| Leaf: attitude at 10-12 leaf stage | 3 | 3 |
| Leaf blade: division (time as for 5) | 1 | 1 |
| Plant: diameter | 5 | 5 |
| Plant: head formation | 3 | 3 |
| Varieties with closed head only: | | |
| Head: degree of overlapping of upper part of | 5 | 5 |
| leaves | | |
| Head: density | 5 | 5 |
| Head: size | 5 | 5 |
| Head: closing of base | 5 | 5 |
| Head: shape in longitudinal section | 1 | 1 |
| Leaf: thickness | 7 | 7 |
| Leaf: attitude at harvest maturity | 3 | 3 |
| Leaf: shape | 1 | 1 |
| Leaf: colour of outer leaves | 2 | 2 |
| Leaf: intensity of colour of outer leaves | 7 | 7 |
| Leaf: anthocyanin coloration | 1 | 1 |
| Leaf: glossiness of upper side | 5 | 5 |
| Leaf: surface profile of outer leaves | 7 | 7 |
| Leaf: blistering | 5 | 5 |
| Leaf: size of blisters | 5 | 5 |
| Leaf blade: degree of undulation of margin | 3 | 3 |
| Leaf blade: presence of incisions on margin on | 1 | 1 |
| apical part | | |
| Leaf blade: venation | 1 | 1 |
| Flowering plant: height | 5 | 5 |
| Axillary sprouting | 1 | 1 |
| Time of harvest maturity | 5 | 5 |
| Time of beginning of bolting under long day | 5 | 5 |
| conditions | | |
| Resistance to downy mildew (Bremia lactucae) | | |
| 1 = susceptible, 9 = resistant. | | |
| Code of the isolate or race: | | |
| NL1 | 9 | 1 |
| NL2 | 9 | 1 |
| NL4 | 9 | 1 |
| NL5 | 9 | 1 |
| NL6 | 9 | 1 |
| NL7 | 9 | 1 |
| NL10 | 9 | 1 |
| NL12 | 9 | 1 |
| NL13 | 9 | 1 |
| NL14 | 9 | 1 |
| NL15 | 9 | 1 |
| BL16 | 9 | 1 |
| BL17 | 1 | 1 |
| Resistance to downy mildew (Bremia lactucae) | | |
| 1 = susceptible, 9 = resistant. Code of the isolate | | |
| or race: | | |
| BL18 | 1 | 1 |
| BL20 | 1 | 1 |
| BL21 | 9 | 1 |
| BL22 | 1 | 1 |
| BL23 | 9 | 1 |
| BL24 | 1 | 1 |
| BL25 | 1 | 1 |
| Resistance to lettuce mosaic virus (LMV) strain | 1 | 1 |
| Ls-1 | | |
| Characteristics additional to the CPVO protocol | | |
| Resistance to root aphids | 1 | 1 |
| Resistance to leaf aphids (Nasonovia ribisnigri) | 9 | 1 |

### EXAMPLE 2

A variety essentially derived from Nasimento is produced by the selection of an induced or naturally occurring mutant plant or off-type plant from plants of Nasimento, which plant retains the expression of the phenotypic characteristics of Nasimento and differs only from Nasimento in the expression of one, or two, or three, or four, or five of the phenotypic characteristics of Nasimento as listed in table 1, when grown side by side with Nasimento on one or two locations in one or two growing seasons.

### EXAMPLE 3

A variety essentially derived from Nasimento is produced by the process of introgression of a trait into plants of the variety Nasimento via the method of recurrent backcrossing and selecting, with or without the aid of molecular markers, the plants which express the characteristics of Nasimento combined with the said trait.

### REFERENCES OF LITERATURE CITED

Briggs, F. N. and P. F Knowles, 1967: “Introduction to Plant Breeding”, Rheinhold Publishing Corporation, New York—Amsterdam—London
Herve Lot et al., 1997: “Coat protein gene-mediated protection in Lactuca sativa against lettuce mosaic potyvirus strains”, Molecular Breeding 3, 75-86.
P. Stam, 2003: “Marker-assisted introgression: speed at any cost?” Proceedings of the Eucarpia Meeting on Leafy Vegetable Genetics and Breeding, Noordwijkerhout, The Netherlands, 19-21 Mar. 2003. Eds. Th. J. L. van Hintum, A. Lebeda, D. Pink, J. W. Schut. P117-124
F. A. van Eeuwijk, and J. R. Law, 2004: “Statistical aspects of essential derivation, with illustrations based on lettuce and barley”, Euphytica 137: 129-137.
Community Plant Variety Office, Boulevard Marechal Foch, FR—49021, Angers Cedex 02: 2001 “CPVO—TP 13/1: Protocol for Distinctness, Uniformity and Stability Tests, Lettuce, Lactuca sativa L. http://www.cpvo.eu.int/documents/TP/vegetales/TP%20lettuce%20151101 .PDF

1. Seed of lettuce variety Nasimento, comprising the characteristics of the seed deposited under NCIMB Accession No. 41293.

2. Plants, or parts thereof, produced by growing the seed of claim 1 and expressing the phenotypic characteristics of said lettuce variety Nasimento, which are the result of its genotype.

3. Leaflets of seedlings produced by seeding the seed of claim 1 at a high density.

4. Seed produced by the plants of claim 2.

5. Plants, or parts thereof, obtained by vegetative reproduction of plants, or parts thereof, of claim 2 expressing the phenotypic characteristics of lettuce variety Nasimento, which are the result of the genotype of Nasimento.

6. A process of sexual or vegetative reproduction of lettuce variety Nasimento.

7. A cell or tissue culture produced from plants or plant parts of claim 2 or 5.

8. A lettuce plant regenerated from the cell or tissue culture of claim 7 expressing the phenotypic characteristics of lettuce variety Nasimento, which are the result of the genotype of Nasimento.

9. A method of producing F1 hybrid lettuce seed comprising the steps of crossing plants of claim 2 with lettuce plants different from Nasimento and harvesting the resultant F1 hybrid lettuce seed.

10. F1 hybrid lettuce seed produced by the method of claim 9.

11. F1 hybrid lettuce plants, or parts thereof, produced by growing the hybrid seed of claim 10.

12. Plants, or parts thereof, obtained by vegetative reproduction of the lettuce plants of claim 11.

13. Seed produced by the F1 hybrid lettuce plants of claim 11 or 12.

14. Plants, or parts thereof, grown from the seed of claim 13.

15. A method of producing F1 lettuce seed comprising the steps of crossing plants of claim 11 or 12 with lettuce plants different from those plants of claim 11 or 12 and harvesting the resultant F1 hybrid lettuce seed.

16. F1 Hybrid lettuce plants, or parts thereof, produced by growing the hybrid seed of claim 13.

17. Plants, or parts thereof, obtained by the vegetative reproduction of the lettuce plants of claim 14.

18. A method of producing essentially derived lettuce plants, comprising the step of predominant derivation from Nasimento by genetic transformation of a desired trait in regenerable plant tissue of claim 7, said essentially derived plants retaining the expression of the phenotypic characteristics of lettuce variety Nasimento,—that are the result of the genotype of Nasimento—, except for those one, or two, or three, or four, or five phenotypic characteristics that have been changed by this method of essential derivation.

19. A method of producing essentially derived lettuce plants, comprising the step of predominant derivation from Nasimento by at least 2 times repeated backcrossing with plants of claim 2 as the recurrent parent, with the aim to introduce a desired trait in Nasimento, and/or to breed a new variety as close as possible to Nasimento, said essentially derived plants retaining the expression of the phenotypic characteristics of lettuce variety Nasimento,—that are the result of the genotype of Nasimento—, except for those one, or two, or three, or four, or five phenotypic characteristics that have been changed by this method of essential derivation.

20. The method of claim 18 wherein said desired trait is selected from the group consisting of male sterility, herbicide resistance, insect resistance, virus disease resistance, nematode resistance, bacterial disease resistance and fungal disease resistance.

21. A method of producing essentially derived lettuce plants, comprising the step of predominant derivation from Nasimento by the selection of a mutant or variant plant out of plants of claim 2 with the aim to change a trait of Nasimento into a desired or different trait and/or to breed a new variety close to Nasimento, and which essentially derived plants retain the expression of the phenotypic characteristics of lettuce variety Nasimento, that are the result of the genotype of Nasimento—, except for those one, or two, or three, or four, or five phenotypic characteristics that have been changed by the method of essential derivation.

22. A method of producing essentially derived lettuce plants, comprising the step of predominant derivation from Nasimento by any breeding method and creating a new variety close to Nasimento, which essentially derived plants retain the expression of the phenotypic characteristics of lettuce variety Nasimento, that are the result of the genotype of Nasimento—, except for those one, or two, or three, or four, or five phenotypic characteristics that have been changed by the method of essential derivation.

23. Plants or parts thereof, obtained by the method of any one of claims 18, 19, 21, or 22 and seed produced by those plants.

24. Plants, or parts thereof, obtained by the method of any one of claims 18, 19, 21, or 22 wherein the plants used in this method are themselves essentially derived from Nasimento by the method of any one of claims 18, 19, 21, or 22 and where the essentially derived plants retain the expression of th phenotypic characteristics of lettuce variety Nasimento, except for those one, or two, or three, or four, or five phenotypic characteristics that have been changed by the method of essential derivation.

25. Seeds produced by the plants of claim 24.

26. Plants, or parts thereof, grown from the seed of claim 25.
